(19) United States
(12) Patent Application Publication
(10) Pub. No.: US 2011/0244006 A1
(43) Pub. Date: Oct. 6, 2011

(54) MICROSPHERE SKIN TREATMENT

(75) Inventors: Jeremy J. Mao, Closter, NJ (US); Eduardo K. Moioli, Coral Springs, FL (US)

(73) Assignee: The Trustees of Columbia University in the City of New York, New York, NY (US)

(21) Appl. No.: 12/745,296

(22) PCT Filed: Nov. 26, 2008
(86) PCT No.: PCT/US08/84901

§ 371 Date: Nov. 30, 2010

Related U.S. Application Data
(60) Provisional application No. 60/990,981, filed on Nov. 29, 2007.

(51) Int. Cl. A61K 8/02; A61K 38/18; A61P 17/02; A61Q 19/08

(52) U.S. Cl. 424/401; 514/7.6; 514/9.1; 424/78.37

(57) ABSTRACT

This application provides a microsphere suitable for tissue engineering that comprises connective tissue growth factor (CTGF). Also provided is a matrix, material or scaffold suitable for tissue engineering that comprises connective tissue growth factor (CTGF) and basic fibroblast growth factor (bFGF). Additionally, methods of treating skin of a human are provided. The methods comprise administering to the skin microspheres comprising a growth factor that increases fibroblast proliferation or collagen, elastin, or glycosaminoglycan synthesis. Further provided are the use of the above microspheres for the treatment of the skin of a human. Additionally, this application proves the use of the above microsphere for the manufacture of a medicament for the treatment of the skin of a human.

### CROSS-REFERENCE TO RELATED APPLICATIONS

This application claims the benefit of U.S. Provisional Application No. 60/990,981, filed Nov. 29, 2007, incorporated by reference herein in its entirety.

### STATEMENT REGARDING FEDERALLY SPONSORED RESEARCH OR DEVELOPMENT

This invention was made with government support under Grant No. R01DE15391 and Grant No. R01EB02332, both awarded by The National Institutes of Health. The government has certain rights in the invention.

### BACKGROUND

The present application generally relates to biological cues for long-lasting skin health and rejuvenation.

Skin aging results in wrinkle (rhytid) formation due to several factors including decreased number of skin (dermal) fibroblasts, decreased fibroblast activity, and decreased dermal extracellular matrix (ECM) proteins such as collagen and elastin fibers as well as proteoglycans and glycosaminoglycans. Additionally, repeated contraction of muscles of facial expression induces wrinkly lines, typically perpendicular to the axis of fiber shortening. Lines of facial expression may become permanent as skin elasticity is decreased with aging. Repeated muscular contractions may also form creases in the facial skin.

There are currently three techniques for skin rejuvenation in the market—facial peel, botulinum toxin injections, and soft tissue fillers.

Facial peels can be achieved by a chemical solution or laser. Chemical peels, which have been in existence for over 30 years, are available in different strengths and are relatively inexpensive.

Laser technology has been performed since the beginning of the 1990's. Although laser treatments are effective, they are very expensive, and require long recovery periods.

Botulinum toxin injections are used for the treatment of facial wrinkles. While there is little or no recovery period involved with this treatment type, it is a temporary solution for the treatment of wrinkles, lasting from 3-4 months at which time repeated treatments are required.

Subcutaneous fillers have been used to correct skin rhytids due to aging or loss of soft tissues. Injectables are highly preferred by patients given that they are relatively quick to administer and minimally discomforting “in office” procedures. Injectable treatment allows precise control of quantity and location of material injection for customizable approaches, and is more convenient for the patient than using pre-shaped implantation materials, which require more invasive procedures. The injectable approaches that have dominated the market for soft tissue fillers include collagen (xenograft, allograft and autograft), freeze-dried acellular dermal tissue, hyaluronic acid, calcium hydroxyapatite spheres and poly-L-lactic acid (PLLA) and polymethylmethacrylate (PMMA) beads. However, the common disadvantage of these procedures is resorption, resulting in need for repeated applications. Static facial rhytids such as in the forehead, glabella, perioral region, and lateral periorbital area respond well to injection filling. Dynamic facial rhytids may be better treated with the application of botulinum toxin, which temporarily paralyzes muscles and does not act a as a filler material. Nevertheless, these treatments still have short activity and there is still a need for repeated injections every few months. Autologous fibroblast injections have also been developed to address facial recontouring, based on their collagen and elastin producing ability; however the injected cells do not retain viability and activity long term, resulting in wrinkle reappearance.

A tensioning polymer useful for tensioning the skin, and a liquid vehicle useful for delivering the tensioning polymer to the skin have also been described (U.S. Patent Application 20060210512). However, this approach is also relatively short lived and requires additional applications.

Fibroblasts (the major cell type in the dermis of the skin), like all cells in the body, are regulated by growth factors. Fibroblast growth factors (FGFs) as well as connective tissue growth factor (CTGF) among others, regulate fibroblast function in the dermis and increase their proliferation and collagen, elastin and glycosaminoglycan synthesis. Current and previous approaches for wrinkle filling only addressed volumetric filling using synthetic materials that do not remodel or materials or cells that are resorbed and lose activity.

Given the need for long-term maintenance of wrinkle filling, these exogenously added growth factors need to be protected against proteolysis that occurs naturally in the body reducing growth factor activity. The present application addresses that need.

### SUMMARY

This application is based on the discovery that a matrix, material or scaffolding that slowly releases certain growth factors is useful for skin administration.

The application is directed to a microsphere suitable for tissue engineering. The microsphere comprises connective tissue growth factor (CTGF).

The application is also directed to a matrix, material or scaffold suitable for tissue engineering. The matrix, material or scaffold comprises connective tissue growth factor (CTGF) and basic fibroblast growth factor (bFGF).

The application is additionally directed to methods of treating skin of a human. The methods comprise administering to the skin microspheres comprising a growth factor that increases fibroblast proliferation or collagen, elastin, or glycosaminoglycan synthesis.

The application is also directed to the use of the microspheres described in this application for the treatment of the skin of a human.

Additionally, this application is directed to the use of a microsphere for the manufacture of a medicament for the treatment of the skin of a human.

### BRIEF DESCRIPTION OF THE DRAWINGS

FIG. 1 is photographs of cultures of primary adult skin fibroblasts after incubation with basic fibroblast growth factor (bFGF) and connective tissue growth factor (CTGF), showing increased extracellular matrix (ECM) production with the growth factor treatments.

FIG. 2 is micrographs of connective tissue stain for skin fibroblasts cultured with bFGF and CTGF showing increased ECM production.

FIG. 3 is a graph showing increased protein synthesis by bFGF-treated primary adult skin fibroblasts.

FIG. 4 is micrographs of primary adult skin fibroblasts treated with bFGF at various doses showing an increase in fibroblast proliferation with increasing bFGF treatment.

FIG. 5 is a graph showing the release curve of bFGF from poly-lactic-co-glycolic (PLGA) microspheres.

FIG. 6 is an illustration of the controlled delivery of bFGF tested in vivo in an immunodeficient mouse model.

FIG. 7 is a graph showing the effects of bFGF released from PLGA microspheres or delivered in solution to constructs (non-encapsulated) on protein synthesis by fibroblasts.

FIG. 8 is a graph of real-time PCR data demonstrating that fibroblasts express several key mRNA markers of fibroblasts.

FIG. 9 is micrographs showing the encapsulation and release of CTGF in PLGA microspheres and microsphere degradation. Panel A shows CTGF-encapsulated PLGA microspheres incorporated in collagen gel. Panel B shows the degradation of the PLGA shell. This degradation was started in the observed 7 days following delivery of CTGF encapsulating PLGA microspheres in collagen gel. Panel C shows the release kinetics of microencapsulated CTGF continuously for the observed 45 days.

FIG. 10 is micrographs and graphs showing CTGF increases collagen and tenascin C production by fibroblasts. Panel A shows fibroblast-like cells without treatment of CTGF. Panel B shows fibroblast-like cells treated with 100 ng/mL CTGF showing increased collagen production per Masson's Trichrome staining Panel C shows that type I collagen production is stimulated by CTGF at 2 wk treatment and 4 wk treatment. CTGF increases collagen production significantly. Panel D shows tenascin C production stimulated by CTGF at 2 wk treatment and 4 wk treatment. CTGF increases tenascin C production significantly.

### DETAILED DESCRIPTION

The inventors have discovered an approach to deliver biologically derived cues via long-term controlled release mechanisms for skin treatment. Growth factors are encapsulated in a matrix, material or scaffold and administered to the skin to promote the proliferation of skin fibroblasts and extracellular matrix. One useful matrix is microspheres, which encapsulate multiple growth factors, protecting them from proteolysis and delivering them in a sustained fashion. See Examples. Additionally, the micron size of the growth factor-loaded spheres allows for injection directly into the dermis in “in office” outpatient procedures.

The application is directed to a microsphere suitable for tissue engineering. The microsphere comprises connective tissue growth factor (CTGF). In some embodiments, the microsphere is biodegradable.

The microsphere of these embodiments can also comprise any other compound, including a second growth factor. In some embodiments, the second growth factor increases fibroblast proliferation or collagen, elastin or glycosaminoglycan synthesis in fibroblasts. An example of such a second growth factor is basic fibroblast growth factor (bFGF).

In some embodiments, CTGF concentration in the microsphere is about 0.0001-10,000,000 ng CTGF/ml microsphere; the concentration may also be about 0.001-100,000 ng/ml, or between about 0.01-1,000 ng/ml, or between about 1-100 ng/ml.

Where other growth factors are present, a useful concentration in the microspheres could be determined by the skilled artisan for any particular application without undue experimentation. When bFGF is present, in some embodiments its concentration is about 0.0001-10,000,000 ng bFGF/ml microsphere; the concentration may also be about 0.001-100,000 ng/ml, or between about 0.01-1,000 ng/ml, or between about 1-100 ng/ml.

Since different microspheres would allow release of a given amount of growth factor at a different rate, it is also useful to measure the “potency” of the growth factor by how much growth factor is released in a given period of time, e.g., a week. In some embodiments, CTGF is released at a rate of about 1 to 1000 ng CTGF/ml microspheres per week, or about 10 to 100 ng/ml, or about 15-50 ng CTGF/ml microspheres per week. Where bFGF is present, in some embodiments, it is released at a rate of about 1 to 1000 ng bFGF/ml microspheres per week, or about 10 to 100 ng/ml, or about 15-50 ng bFGF/ml microspheres per week.

The microspheres of this application can comprise any material considered to be suitable for tissue engineering. The skilled artisan could identify without undue experimentation a suitable microsphere for any purpose as to material, size, density, or any other physical characteristic. In some embodiments, the microsphere comprises a natural polymer. Nonlimiting examples of useful natural materials include collagen, gelatin, fibrin, and lysosome. In other embodiments of this invention, the microsphere comprises a synthetic polymer. Nonlimiting examples of useful synthetic polymers include poly(dl-ε-caprolactone), poly(lactic-coglycolic) acid (PLGA), poly(D,L-lactide) (PLA), poly-L-lactic acid (PLLA), a polyanhydride, and a chitosan. In some embodiments of the present invention, the synthetic polymer is PLGA. The microsphere can also comprise both a natural polymer and a synthetic polymer.

The microspheres of these embodiments are not narrowly limited to any particular diameter of microsphere. It is envisioned that the most useful size range of microspheres is about 0.002 to about 2,000 μm. In some embodiments, and as practiced in the microsphere work described in the Examples, a microsphere of the instant application has a diameter of about 108 μm. In additional embodiments, the microsphere comprises PLGA and has a diameter of about 108 μm.

The present application is also directed to a matrix, material or scaffold suitable for tissue engineering. The matrix, material or scaffold comprises connective tissue growth factor (CTGF) and basic fibroblast growth factor (bFGF).

As used herein, a “matrix” is an amorphous structure, e.g., a gel, in which the growth factors are suspended. A “material” is a fibrous composition, and a “scaffold” has tertiary structure, e.g., a columnar structure or a porous structure such as in a typical microsphere, e.g., with fairly uniform pores in which, in some embodiments of the present application, a growth factor solution permeates. The invention is not limited to any particular matrix, material or scaffold. The matrix, material or scaffold may be biodegradable. In various embodiment, the matrix, material or scaffold is any of the above-described microspheres that include bFGF.

There are several potential applications for the above-described matrix, material or scaffold. One useful application is for skin rejuvenation or repair. Another useful application of the matrix, material or scaffold is for promoting the regeneration of other fibrous tissues such as periodontal ligament, tendons, burns, interstitial tissue, and ligaments.

The application is also directed to a method of treating skin of a human. The method comprises administering to the skin microspheres comprising a growth factor that increases fibroblast proliferation or collagen, elastin, or glycosaminoglycan synthesis.

Any growth factor that increases fibroblast proliferation or collagen, elastin, or glycosaminoglycan synthesis can be used for these methods. In some embodiments, the growth factor is bFGF or CTGF. For example, the microspheres used in these methods can comprise both bFGF and CTGF. Additionally, any of the above-described microspheres can include bFGF. In some embodiments, the microspheres comprise a synthetic polymer, for example PLGA.

These methods can be used on unblemished skin, to increase the ECM in the treated skin. The methods can also be used on skin needing repair. In some embodiments, the microspheres are injected into the dermis of the skin at or near a wrinkle of the skin. In other embodiments, the microspheres are injected into the dermis of the skin at or near a pock mark of the skin. In additional embodiments, the microspheres are injected into the dermis of the skin at or near a burn of the skin. In further embodiments, the microspheres are injected into the dermis of the skin at or near a scar of the skin. In still further embodiments, the microspheres are injected into the dermis of the skin at or near a defect of the skin. Non-limiting examples of defects include skin that was removed due to cancer, infection or trauma.

In some embodiments of these methods, the treated skin produces increased tenascin C, when compared to untreated skin. See Example 9. Tenascin C is an extracellular matrix glycoprotein that is abundant in developing tendons, bone and cartilage. It is transiently expressed upon tissue injury and is localized to the wound edge, where it promotes fibroblast migration in tissue repair (Trebaul et al., 2007). Without being bound by any particular mechanism, the increased tenascin C levels upon treatment with CTGF indicate that CTGF treatment induces tissue repair mechanisms.

The application is further directed to the use of any of the above-described microspheres for the treatment of the skin of a human. Some of the microspheres in these embodiments comprise a synthetic polymer, for example PLGA. In various embodiments, both CTGF and bFGF are in the microspheres and the microspheres are about 108 μm in diameter. For these uses, the microsphere can be injected into the dermis of the skin at or near a wrinkle, a pock mark, a burn, a scar or a defect of the skin.

The application is additionally directed to the use of any of the above-described microspheres for the manufacture of a medicament for the treatment of the skin of a human. Various microspheres of these embodiments comprise a synthetic polymer, for example PLGA. In some aspects, both CTGF and bFGF are in the microspheres and the microspheres are about 108 μm in diameter. In some embodiments, the treatment is the injection of the microsphere into the dermis of the skin at or near a wrinkle, a pock mark, a burn, a scar or a defect of the skin.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1

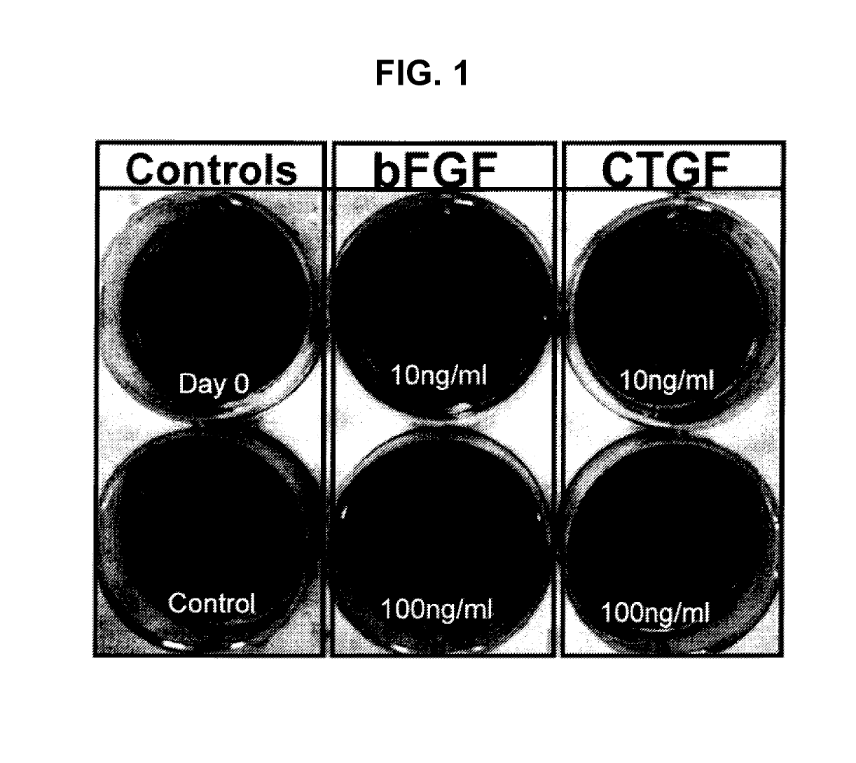

This example illustrates the effects of basic fibroblast growth factor (bFGF) and connective tissue growth factor (CTGF) on extracellular matrix production (ECM) by primary adult skin fibroblasts. bFGF or CTGF was added to cultures of adult skin fibroblast for seven days. Shown in FIG. 1 are cultures of adult skin fibroblasts following seven days incubation with 10 ng/ml or 100 ng/ml of bFGF, or CTGF compared to controls at both day 0 and day 7. Results are shown following staining of the cultures with Mason's Trichrome staining Increases in extracellular matrix production by primary adult skin fibroblasts was evident following 7 days culture with 10 ng/ml or 100 ng/ml bFGF or CTGF.

### Example 2

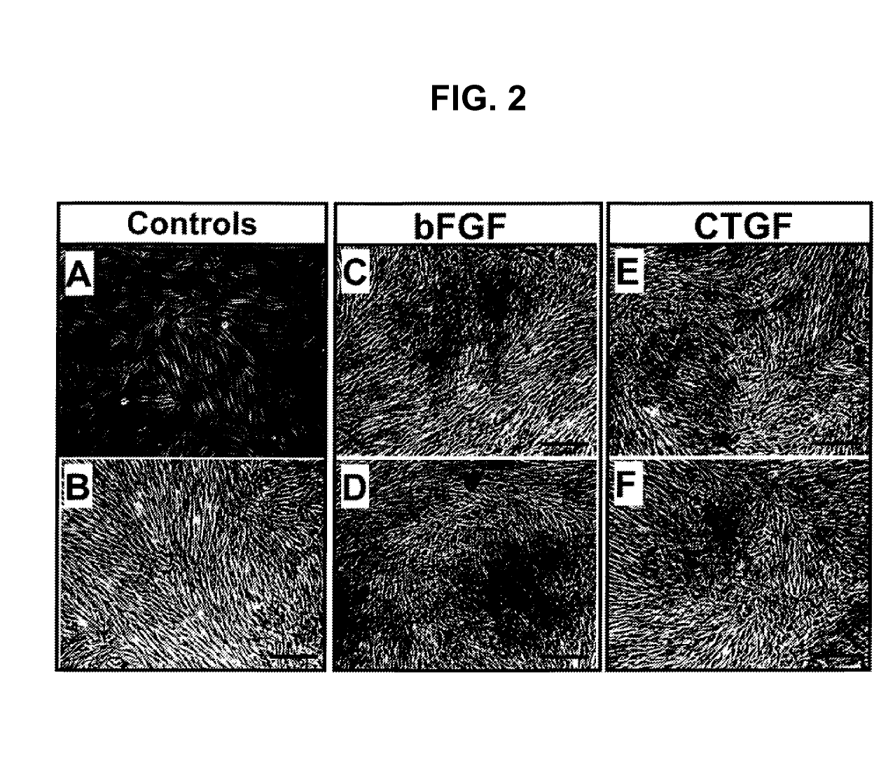

This example illustrates the effects of bFGF and CTGF on the production of ECM by skin fibroblasts. Shown in FIG. 2 are skin fibroblasts cultured with or without (controls) bFGF and CTGF and stained with a connective tissue stain. Treatment with the growth factors show increased ECM production.

### Example 3

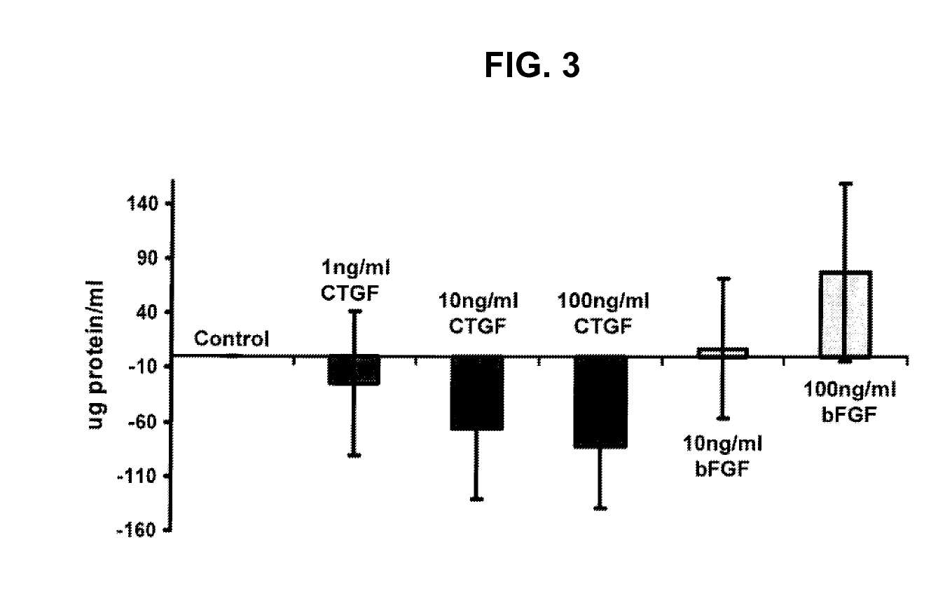

This example illustrates the effects of bFGF on protein synthesis by primary adult skin fibroblasts. FIG. 3 shows protein synthesis (μg protein/ml) in primary adult skin fibroblasts incubated with 1 ng/ml, 10 ng/ml, or 100 ng/ml of CTGF, or with 10 ng/ml or 100 ng/ml of bFGF. Results of the study indicate that bFGF at 100 ng/ml caused an increase in protein synthesis. Protein synthesis was decreased in cultures of fibroblasts following incubation with all three concentrations of CTGF tested.

### Example 4

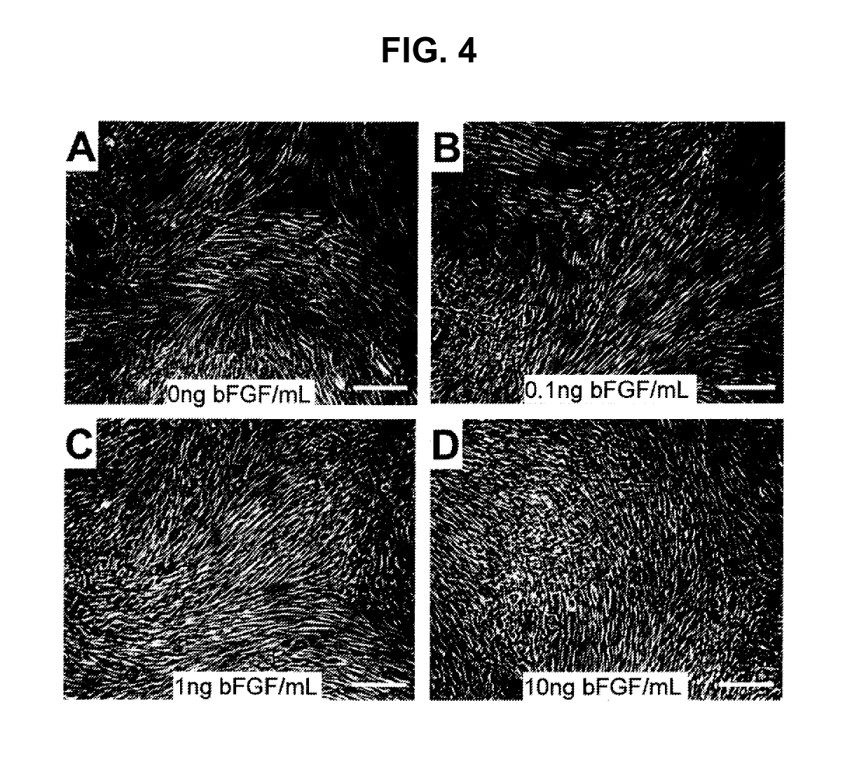

This example illustrates the effects of bFGF on the proliferation of primary adult skin fibroblasts following incubation with bFGF at various concentrations. FIG. 4 shows the effects on cell proliferation following incubation with bFGF at concentrations of A) 0 ng/ml, B) 0.1 ng/ml, C) 1 ng/ml or D) 10 ng/ml. The photographs show a dose dependent increase in cell proliferation increasing with an increase in bFGF concentration.

### Example 5

To address the shortcomings of rapid denaturation and diffusion of growth factors and other biological cues that are delivered in vivo, a method was developed for using tissue engineering scaffolding to cause a long-term release of the growth factor. bFGF was encapsulated in poly-lactic-co-glycolic (PLGA) microspheres. PLGA is biocompatible and FDA approved. The micron size of spheres allows for injectable, minimally discomforting “in office” procedures.

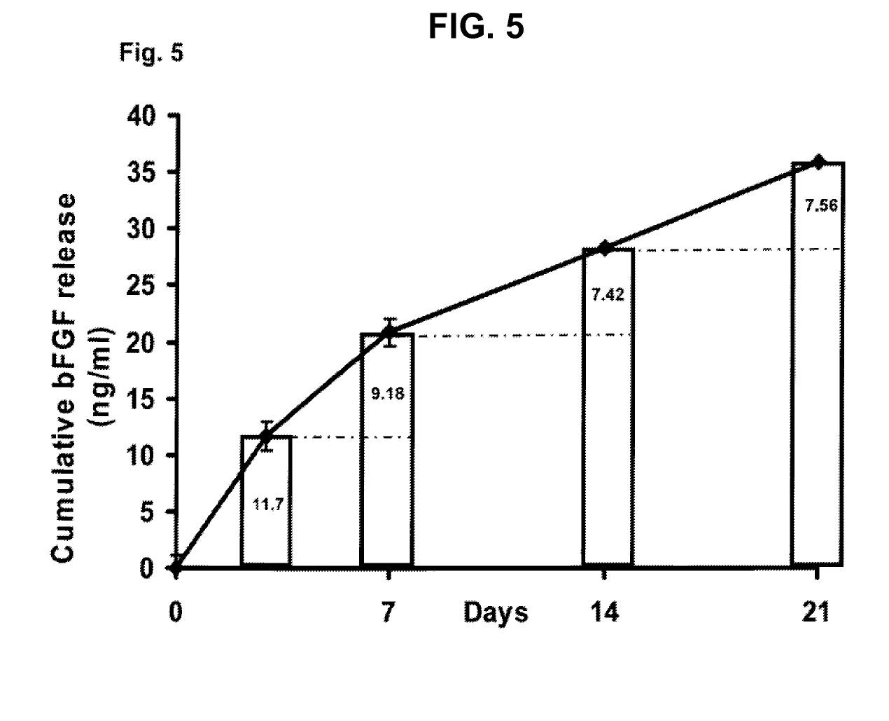

Shown in FIG. 5 are the results of the cumulative release of bFGF (ng/ml) as a function of time (0, 3, 7, 14, and 21 days). This release curve shows that microencapsulated bFGF is released for up to the tested 21 days.

### Example 6

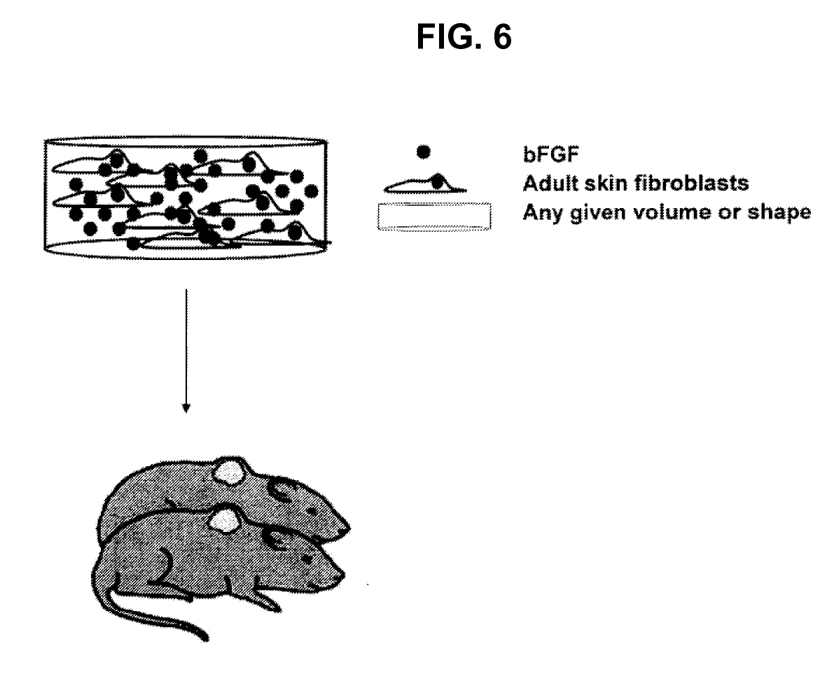

As diagramed in FIG. 6, the fibroblast-rejuvenating effects of controlled delivery of bFGF were tested in vivo in an immunodeficient mouse model, where human skin derived fibroblasts were seeded in biocompatible 3D constructs and injected with bFGF loaded PLGA microspheres for sustained delivery.

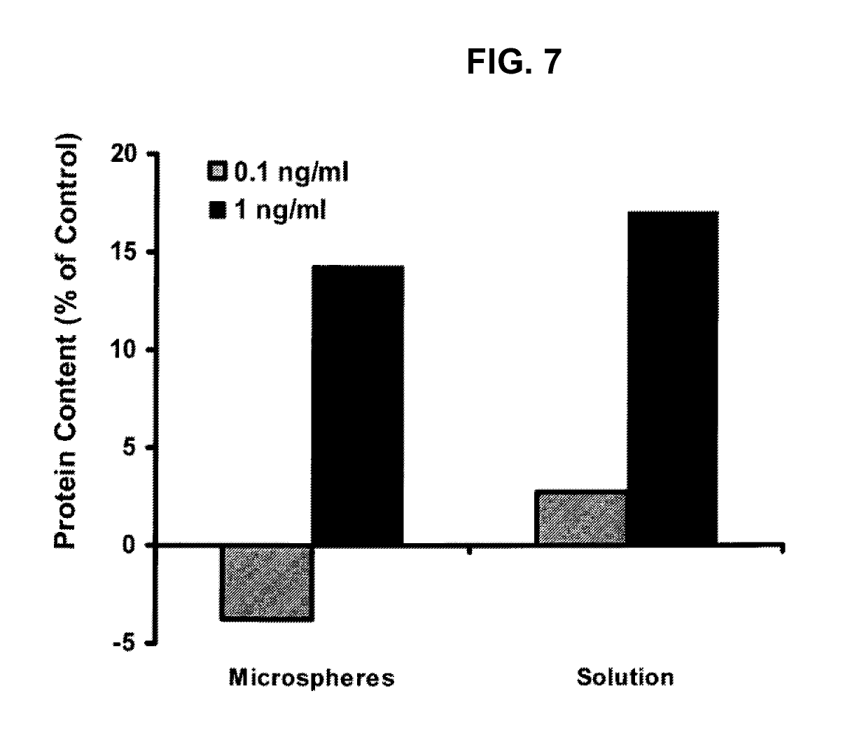

bFGF, released from PLGA microspheres as well as delivered in solution to the constructs (non-encapsulated) resulted in increased protein synthesis by fibroblasts. In FIG. 7, protein content (% of control) is shown as a function of either microspheres or solution, with concentrations of bFGF of 0.1 ng/ml or 1 ng/ml. Results show that protein content is significantly increased with 1 ng/ml of bFGF, with both microspheres and with solution.

### Example 7

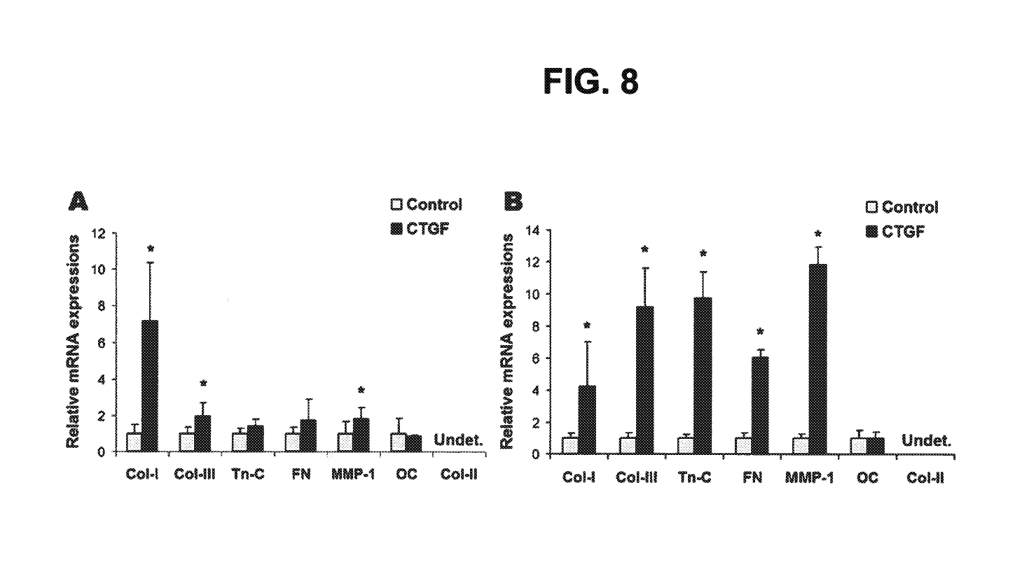

In this example, real-time PCR data is used to show that fibroblasts express several key mRNA markers of Fibroblasts. See FIG. 8.

Relative mRNA expression of type I collagen, type III collagen, tenascin C, fibronectin, MMP-1, osteopontin and type II collagen was measured in fibroblasts following incubation with, or without, CTGF for 2 (Panel A—FIG. 8) or 4 (Panel B) weeks.

At 2 weeks of culture with CTGF treatment, type I collagen, type III collagen and MMP-1 expression was significantly higher than controls. Negligible expression of osteopontin and type II collagen indicate that fibroblasts do not synthesize bone and cartilage markers. By 4 wks of culture with CTGF, tenascin C and fibronectin, expression significantly increased. Increases in mRNA expression of type I collagen, type III collagen and MMP-1 remain significant. This further establishes that CTGF augments the synthesis of extracellular matrix molecules by fibroblasts.

### Example 8

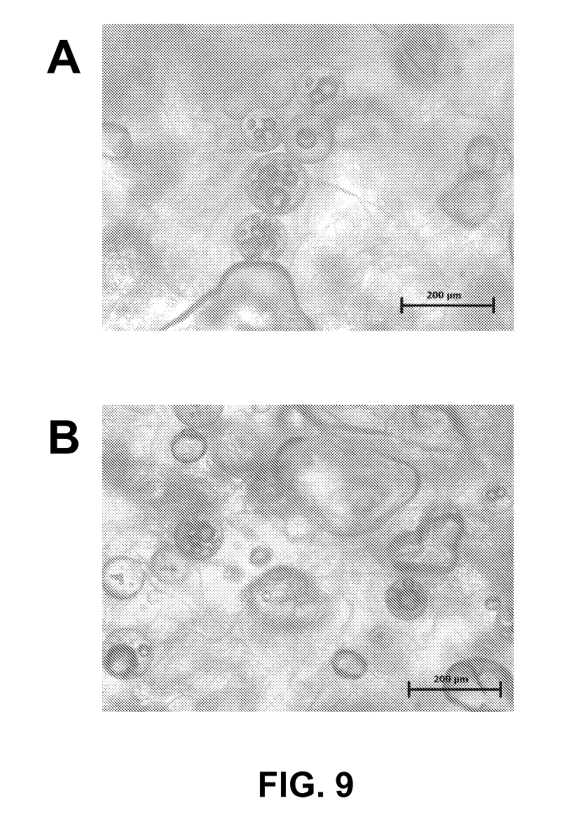

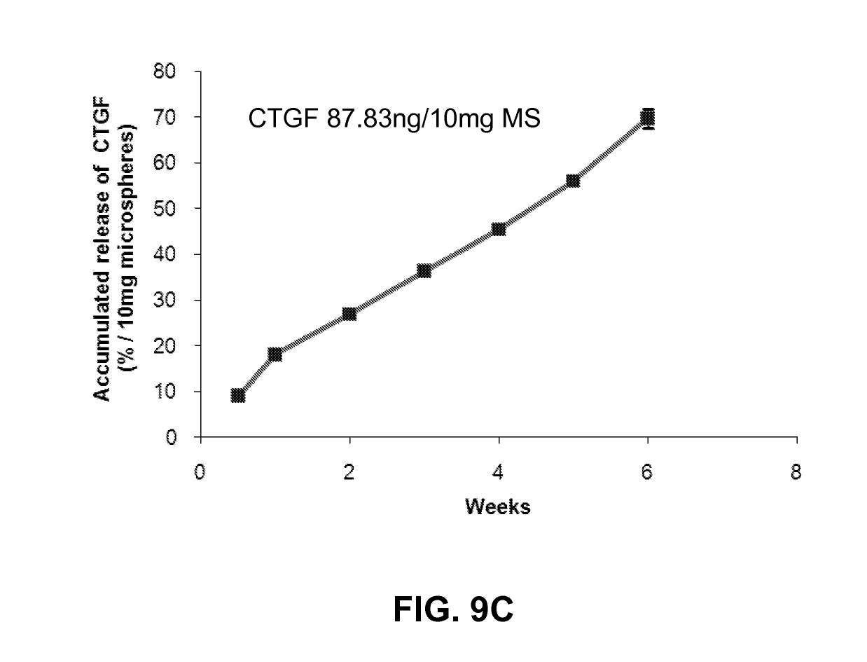

In this example, CTGF release and microsphere degradation is shown. CTGF (87.83 ng) was encapsulated in 10 mg PLGA microspheres. FIG. 9A is a micrograph showing CTGF-encapsulated PLGA microspheres incorporated in collagen gel. After 7 days, there was significant degradation of the PLGA shell (FIG. 9B). The release of CTGF from the microspheres was nearly linear for 45 days (FIG. 9C).

### Example 9

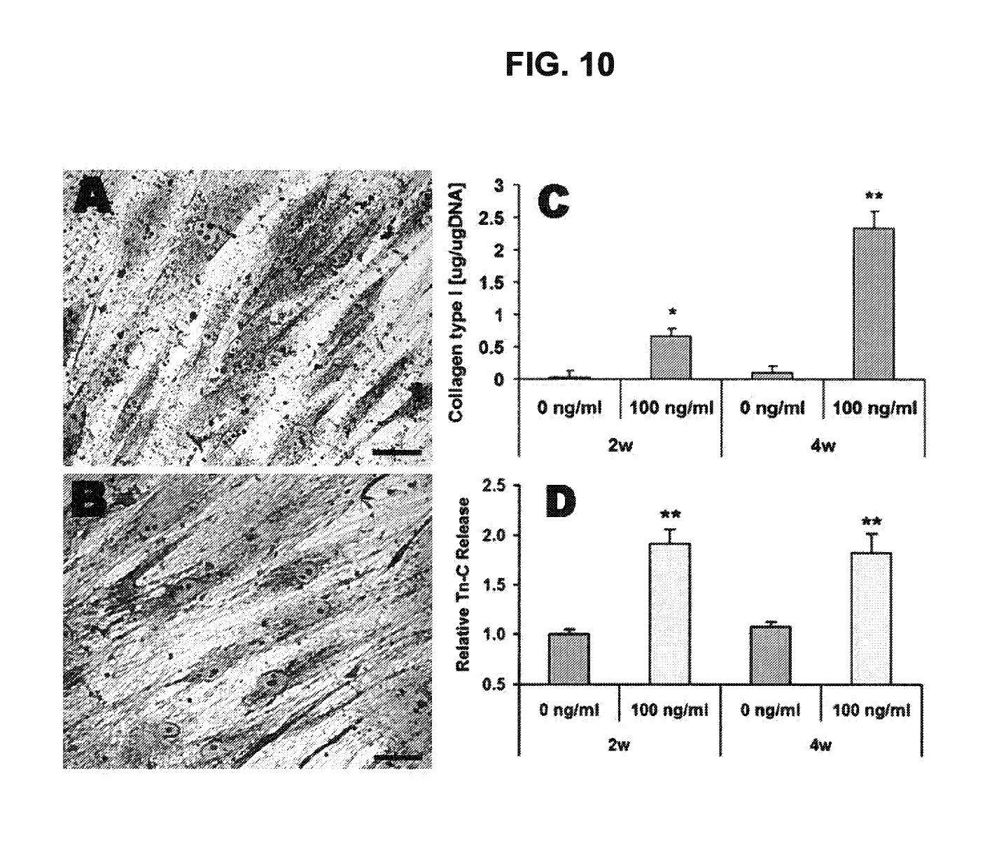

In this example, effects of CTGF on parameters of skin formation were studied. Fibroblast-like cells were treated with 100 ng/ml CTGF and collagen and tenascin C production was observed. FIG. 10A shows fibroblast-like cells without treatment of CTGF. By contrast, FIG. 10B shows fibroblast-like cells treated with 100 ng/ml CTGF, showing increased collagen production per Masson's Trichrome staining FIG. 10C is a graph showing that type I collagen production is stimulated by CTGF at 2 wk treatment and 4 wk treatment. Thus, CTGF increases collagen production significantly. FIG. 10D shows that tenascin C production is stimulated by CTGF 2 wks and 4 weeks after treatment. Thus, CTGF increases tenascin C production significantly.

### REFERENCES

Burney, K. S. 2003, Evaluation of sustained release of antisense oligonucleotide from poly DL (lactide-co-glycolide) microspheres targeting fibrotic growth factors CTGF and TGF-β1. Masters Thesis, University of Florida.
Tabata, Y. 2006, Potential of drug delivery technology in tissue regeneration therapy. J. Hard Tissue Biol. 15:73-81.
Trebaul, A. et al. 2007, Regulation of fibroblast migration by tenascin-C. Biochem. Soc. Trans. 35:695-697.
U.S. Patent Application Publication No. 2006/0240056.
U.S. Patent Application Publication No. 2006/0073178.
U.S. Patent Application Publication No. 2006/0039896.
U.S. Pat. No. 5,770,209.
U.S. Pat. No. 5,801,192.
U.S. Pat. No. 5,837,258.
U.S. Pat. No. 5,976,878.
U.S. Pat. No. 6,582,960.
U.S. Pat. No. 6,696,073.
U.S. Pat. No. 6,699,287.
U.S. Pat. No. 6,719,970.
U.S. Pat. No. 6,852,331.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1-41. (canceled)

42. A method of treating skin of a subject comprising:
administering to skin of a subject a microsphere comprising at least one growth factor;
wherein the at least one growth factor increases fibroblast proliferation; collagen synthesis; elastin synthesis; or glycosaminoglycan synthesis.

43. The method of claim 42, wherein the microsphere comprises connective tissue growth factor (CTGF).

44. The method of claim 42, wherein the microsphere comprises basic fibroblast growth factor (bFGF).

45. The method of claim 42, wherein the microsphere comprises connective tissue growth factor (CTGF) and basic fibroblast growth factor (bFGF).

46. The method of claim 42, wherein administering to skin of a subject a microsphere comprising at least one growth factor comprises:
administering a first microsphere comprising basic fibroblast growth factor (bFGF); and
administering a second microsphere comprising connective tissue growth factor (CTGF).

47. The method of claim 45, wherein the microsphere comprises:
(i) between about 0.0001 and about 10,000,000 ng CTGF/ml microspheres and between about 0.0001 and about 10,000,000 ng bFGF/ml microspheres;
(ii) between about 0.001 and about 100,000 ng CTGF/ml microspheres and between about 0.001 and about 100,000 ng bFGF/ml microspheres;
(iii) between about 0.01 and about 1,000 ng CTGF/ml microspheres and between about 0.01 and about 1,000 ng bFGF/ml microspheres; or
(iv) between about 1 and about 100 ng CTGF/ml microspheres and between about 1 and about 100 ng bFGF/ml microspheres.

48. The method of claim 45, wherein the microsphere releases:
(i) between about 1 and about 1000 ng CTGF/ml microspheres per week and between about 1 and about 1000 ng bFGF/ml microspheres per week.
(ii) between about 10 and about 100 ng CTGF/ml microspheres per week and between about 10 and about 100 ng bFGF/ml microspheres per week; or
(ii) between about 15 and about 50 ng CTGF/ml microspheres per week and between about 15 and about 50 ng bFGF/ml microspheres per week.

49. The method of claim 42, wherein the microsphere comprises a synthetic polymer.

50. The method of claim 49, wherein the microsphere comprises a synthetic polymer selected from the group consisting of poly(dl-c-caprolactone), poly(lactic-coglycolic) acid (PLGA), poly(D,L-lactide) (PLA), poly-L-lactic acid (PLLA), a polyanhydride, and a chitosan.

51. The method of claim 50, wherein the microsphere comprises poly(lactic-coglycolic) acid (PLGA).

52. The method of claim 42, wherein the microsphere comprises a natural polymer.

53. The method of claim 52, wherein the microsphere comprises a natural polymer selected from the group consisting of collagen, gelatin, fibrin, or lysosome.

54. The method of claim 42, wherein the microsphere comprises a natural polymer and a synthetic polymer.

55. The method of claim 42, wherein the microsphere is a biodegradeable microsphere.

56. The method of claim 42, wherein the microsphere has a diameter of about 0.002 to about 2,000 μm.

57. The method of claim 42, wherein the microsphere comprises PLGA and has a diameter of about 108 μm.

58. The method of claim 42, wherein the microsphere is injected into dermis of the skin at or near: (i) a wrinkle of the skin; (ii) a pock mark of the skin; (iii) a burn of the skin; (iv) a scar of the skin; or (v) a defect of the skin.

59. The method of claim 42, wherein increased levels of tenascin C is produced by the skin when compared to untreated skin.

60. A microsphere suitable for tissue engineering, comprising connective tissue growth factor (CTGF); basic fibroblast growth factor (bFGF); or CTGF and bFGF.

61. A matrix, material or scaffold suitable for tissue engineering, comprising a micrsosphere suitable for tissue engineering, the microsphere comprising connective tissue growth factor (CTGF); basic fibroblast growth factor (bFGF); or CTGF and bFGF.
